(19) United States
(12) Patent Application Publication
(10) Pub. No.: US 2008/0085287 A1
(43) Pub. Date: Apr. 10, 2008

(54) Food Additve

(75) Inventors: Eric Auclair, Avelin (FR); David Kalkhoven, Lille (FR); Pascal Termignon, Marcq en Baroeul (FR); Laurent Michel Bonanno, Champigny Sur Marne (FR); Eric Oriol, Champigny Sur Marne (FR)

Correspondence Address: SUGHRUE MION, PLLC, 2100 PENNSYLVANIA AVENUE, N.W., SUITE 800, WASHINGTON, DC, 20037, US

(73) Assignee: LESAFFRE ET COMPAGNIE, Paris (FR)

(21) Appl. No.: 11/660,273

(22) PCT Filed: Aug. 17, 2005
(86) PCT No.: PCT/FR05/02095

§ 371 Date: Feb. 16, 2007

(30) Foreign Application Priority Data
Aug. 17, 2004 (EP) 04 019460.7

(51) Int. Cl. A61K 36/06

(52) U.S. Cl. 424/195.16

(57) ABSTRACT

Agent containing a yeast derivative comprising 5′-nucleotides and/or peptides of at least 1000 daltons useful for the prevention and/or treatment of cannibalism in livestock, the preparation and the use thereof.

The invention relates to the prevention and/or treatment of cannibalism in livestock. Today's modern industrial livestock breeders endeavour to produce the largest amounts of meat, milk and eggs rapidly and inexpensively, in tight production areas. In some of these intensive factory farms, syndromes such as cannibalism have been observed. The term “cannibalism”, as employed herein, is not restricted to the case, very rare in practice, of one animal eating another. In the present context, the term “cannibalism” refers to any incident in which one animal injures another animal by biting it. Hence the term “cannibalism” encompasses the pecking observed in poultry farms and among laying hens and game birds, and the caudophagy (tail and/or ear biting) sometimes seen in pig farms, as well as aggressive behavior in ruminants.

Cannibalism such as this is detrimental to animal welfare and lowers yields. Some commercial products aimed at improving animal well-being are available on the market. For example, in 2000, the veterinary laboratory Ceva Santé Animale commercialized a pheromone under the name Suilence®, which aims to reduce caudophagy in pig farms. Ascorbic acid has also been used as a feed additive to reduce cannibalism in livestock.

The present invention relates to the observation that, in a surprising manner, it is possible to prevent and/or treat (reduce) cannibalism in livestock by administering to said animals a yeast derivative containing 5′-nucleotides and/or peptides, said peptides having a molecular weight of at least 1000 daltons, preferably from 1000 to 10,000 daltons.

Patent U.S. Pat. No. 5,188,851 relates to the addition of 5′-nucleotides to a feed composition in order to improve the appetability of farmed fish.

Patent U.S. Pat. No. 3,686,392 relates to the addition of 5′-nucleotides to the feed of a range of livestock in order to accelerate the growth rate and improve the feed conversion from feed to weight.

The two patents U.S. Pat. No. 6,248,716 and U.S. Pat. No. 6,184,208 propose numerous peptides for modulating physiological processes, such as weight gain, epithelium and hair growth, wound healing, and for stimulating reparative and anabolic processes, as analgesics and as sedatives.

In patent application WO02/067959, peptides of molecular weight less than 10,000 daltons are extracted from yeast subjected to a physical or chemical stress prior to autolysis. Said peptides are used in humans in particular as stress-reducers, sedatives and neurotrophic factor.

The invention relates to a method for preventing and/or reducing cannibalism in livestock comprising administering a yeast derivative containing 5′-nucleotides and/or peptides having a molecular weight of at least 1000 daltons, preferably from 1000 to 10,000 daltons. Preferably, the invention relates to a method for preventing and/or reducing cannibalism in livestock comprising administering 5′-nucleotides and/or peptides having a molecular weight of at least 1000 daltons, preferably 1000 to 10,000 daltons.

The nucleotides are nucleoside phosphoric esters, imperatively comprising:
a purine or pyrimidine nitrogen base,
a pentose:ribose or deoxyribose, and
one or more phosphoric acid molecules.

In the spirit of the invention, the term “5′-nucleotides” includes 5′-nucleotides as such, as well as the salts, hydrates, hydrated salts, and other physiologically acceptable forms thereof. In particular, the 5′-nucleotides comprise at least one 5′ phosphate group.

In the spirit of the invention, the term “peptide” includes peptides as such, as well as the salts and other physiologically acceptable forms thereof.

According to the invention, the 5′-nucleotides are or advantageously comprise ribonucleotides.

In a useful manner, the 5′-nucleotides, and in particular the 5′-ribonucleotides, are or comprise 5′-nucleotide monophosphates.

Preferably they are or comprise mononucleotides.

In particular the 5′-nucleotides can be or comprise 5′-IMP (inosine 5′-monophosphate), 5′-GMP (guanosine 5′-monophosphate), 5′-AMP (adenosine 5′-monophosphate), 5′-UMP (uridine 5′-monophosphate) or 5′-CMP (cytidine 5′-monophosphate). Said 5′-nucleotides are preferably used as a combination of at least two of said 5′-nucleotides, more preferably in the form of a combination of three of said 5′-nucleotides, and even more preferably a combination of four or five of said 5′-nucleotides. They can comprise for example a combination 5′-IMP with a 5′-nucleotide selected from the group consisting of 5′-GMP, 5′-AMP, 5′-UMP and 5′-CMP; or else a combination of 5′-GMP with a 5′-nucleotide selected in the group consisting of 5′-AMP, 5′-UMP and 5′-CMP; a combination of 5′-AMP with 5′-UMP or 5′-CMP; or else a combination of 5′-UMP with 5′-CMP. They can also comprise a combination of 5′-IMP with one of the combinations from the group consisting of: 5′-GMP and 5′-AMP, 5′-GMP and 5′-UMP, 5′-GMP and 5′-CMP, 5′-AMP and 5′-UMP, 5′-AMP and 5′-CMP, 5′-UMP and 5′-CMP. They can also comprise a combination of 5′-GMP with a combination from the group consisting of: 5′-AMP and 5′-UMP, 5′-AMP and 5′-CMP, and 5′-UMP and 5′-CMP. They can also comprise a combination of four of said 5′-nucleotides or else a combination of five of said 5′-nucleotides. Preferably they comprise a combination of 5′-GMP, 5′-UMP, 5′-CMP and 5′-AMP; or a combination of 5′-GMP, 5′-UMP, 5′-CMP and 5′-IMP; or else a combination of 5′-GMP, 5′-UMP, 5′-CMP, 5′-IMP and 5′-AMP. The 5′-nucleotides can be obtained by different methods of production and synthesis. For the invention, the 5′-nucleotides, and in particular the flavour enhancers 5′-GMP and 5′-IMP, can be obtained by any one of said methods.

In a preferred embodiment of the invention, the yeast derivative, the 5′-nucleotides and/or the peptides originate, at least partially and preferably entirely, from yeast cells, more preferably from cells of yeast of the genus Candida or the genus Saccharomyces. Preferably, said yeast cells belong to the species Saccharomyces cerevisiae or to the species Candida utilis (generally, species of Candida used for food are commonly called Torula).

The invention therefore also relates to providing livestock with a yeast derivative, and in particular a yeast derivative containing 5′-nucleotides and/or peptides having a molecular weight of at least 1000 daltons, preferably from 1000 to 10,000 daltons, in particular for the prevention and/or treatment of cannibalism in livestock. Notably, the yeast derivative can be an enzymatic hydrolysate of yeast and, in particular, a yeast extract, such as a yeast extract resulting from an enzymatic hydrolysate of yeast by elimination of non-water soluble materials. The peptides of molecular weight of at least 1000 daltons, preferably from 1000 to 10,000 daltons, derived from yeast are preferably from a yeast derivative, preferably in the form of an enzymatic hydrolysate of yeast, and even more preferably in the form of a yeast extract.

The 5′-nucleotides 5′-GMP and 5′-IMP are known for their particularly noteworthy flavour enhancing properties. In particular it is known how to obtain yeast extracts having a high content of said 5′-nucleotides and therefore a flavour enhancer effect by methods adapted for the production of yeast extracts.

In fact, in conventional methods for the production of yeast extracts which comprise autolysis or enzymatic hydrolysis, mainly by endogenous yeast enzymes, the yeast ribonucleic acid (RNA) is mainly decomposed into tasteless nucleotides such as adenosine 3′-monophosphate (3′-AMP) and guanosine 3′-monophosphate (3′-GMP), bases, riboses and phosphates. Said endogenous yeast enzymes are mainly ribonucleases, specific 3′-nucleotidases, acid phosphatases. Said conventional methods do not therefore yield yeast extracts rich in 5′-nucleotides.

Suitable methods for obtaining yeast derivatives rich in 5′-nucleotides are described in the reference work “Yeast Technology” by G. Reed and T. W. Nagodawithana, 2nd edition (Van Nostrand Reinhold, ISBN 0-442-31892-8) pages 382 to 385. In particular it is known how to prepare yeast extracts rich in 5′-nucleotides by enzymatic hydrolysis of yeasts in the presence of 5′-phosphodiesterase with deactivation of endogenous yeast phosphatases and nucleases. This yields yeast extracts containing the following 5′-nucleotides: 5′-GMP, 5′-UMP, 5′-CMP and 5′-AMP (corresponding to the four RNA bases). In contrast to 5′-GMP, 5′-AMP is not an efficient flavour enhancer. However, 5′-AMP can be converted into the flavour enhancer 5′-IMP by addition of AMP deaminase. This yields a yeast extract containing 5′-GMP, 5′-UMP, 5′-CMP, 5′-IMP, and possibly some residual 5′-AMP. Among the known methods adapted for the production of yeast derivatives rich in 5′-nucleotides and/or peptides having a molecular weight of at least 1000 daltons, and preferably from 1000 to 10,000 daltons, the following methods may also be mentioned:

Patent U.S. Pat. No. 4,810,509 describes a method for producing yeast extracts rich in 5′-nucleotides comprising (1) a step of heating a yeast suspension between 55° C. and 70° C., (2) a step of yeast cell autolysis at pH 8 to 10, (3) adjustment of the pH of the autolysed yeast suspension to between 5 and 7, (4) a step of heating said suspension at 90° C. or above, (5) elimination of insoluble material from said heated suspension, and (6) recovery of the yeast extract containing 5′-nucleotides. Treatment with an enzyme 5′-adenylate deaminase (AMP deaminase) after autolysis enables to convert the 5′-AMP into 5′-IMP which is more odorous therefore more sought-after.

The method disclosed in EP-A-0299078 consists in heating a yeast suspension containing a large amount of RNA between 80° C. and 120° C. (to destroy ribonucleases), then extracting the RNA with an alkaline treatment and cutting it into 5′-nucleotides through the action of a 5′-phosphodiesterase, and optionally a deaminase, so as to obtain an extract rich in 5′-GMP and 5′-IMP.

The method disclosed in WO02/067959 consists in preparing a yeast derivative by autolysis at a temperature of more than 35° C., for example comprised between 35° C. and 70° C., preferably between 50° C. and 60° C. The yeasts are preferably hydrolysed during or after autolysis with one or more proteases. Optionally, the product can be centrifuged and an additional supernatant ultrafiltration step can be carried out. In this way it can be used for the prevention and/or treatment of cannibalism in livestock. Other classical methods can be used to purify the supernatant, in particular chromatographic purification methods.

The invention relates in particular to the prevention and/or treatment of cannibalism in livestock by administering to said livestock a yeast derivative, said yeast derivative possibly being an enzymatic hydrolysate of yeast containing non-water-soluble membranes and/or membrane fragments of yeast cells, or a yeast extract obtained from an enzymatic hydrolysate of yeast by elimination of said membranes and membrane fragments, and in particular one such yeast derivative containing 5′-nucleotides and/or peptides having a molecular weight of at least 1000 daltons, preferably from 1000 to 10,000 daltons. Such yeast derivatives can be obtained in particular by the foregoing methods promoting the formation of 5′-nucleotides.

In a useful manner, the yeast derivatives have a degradation rate expressed as the ratio of amino N/total N, which is less than or equal to 40%, preferably from 25% to 40%. “N” is the symbol for nitrogen. “Total N” is the nitrogen content determined by the Kjeldahl method and “amino N” is the nitrogen content determined by Sørensen's formol titration of nitrogen assay.

The yeast derivatives containing 5′-nucleotides and/or peptides of at least 1000 daltons, and in particular from 1000 to 10,000 daltons, are preferably derivatives of Candida or Saccharomyces, and more preferably derivatives of Saccharomyces cerevisiae or Candida utilis.

The yeast is preferably a baker's yeast. A baker's yeast is a yeast belonging to the genus Saccharomyces, produced essentially by multiplication or aerobic culture as in the teachings of chapter 6 “Baker's yeast production” of the reference work “Yeast Technology” cited earlier.

The yeast can also be a brewer's yeast, a wine yeast or a distillery yeast, which are subproducts of beer, wine and alcohol production, respectively and which have therefore been used in the production of beer, wine or alcohol prior to being recovered to be made into a yeast derivative containing 5′-nucleotides and/or peptides having a molecular weight of at least 1000 daltons.

The yeast derivatives advantageously have a 5′-nucleotide content based on dry matter of at least 2% by mass, preferably at least 3% by mass, more preferably at least 4% by mass and even more preferably at least 5% by mass, and/or a content in peptides of molecular weight of 1000 to 10,000 daltons, based on dry matter of at least 4% by mass, preferably at least 6% by mass and even more preferably at least 8% by mass.

According to the invention, the yeast derivatives containing 5′-nucleotides and/or peptides having a molecular weight of at least 1000 daltons can have a dry matter content of at least 90% by mass, preferably at least 94% by mass and even more preferably at least 96% by mass.

According to a particularly preferred embodiment, the yeast derivative contains 6.8% of 5′-nucleotides, at least 6% of peptides of molecular weight of 1000 to 10,000 daltons and has a total N content determined by the Kjeldahl method of approximately 11.3%, an amino N content determined by Sørensen formol assay of approximately 4.1% and a protein content (6.25× nitrogen) of approximately 70.6%. Said product, commercialized by Bio Springer (94701 Maisons-Alfort, France), is described in further detail in Table 1.

The invention is effective for the prevention and/or treatment of cannibalism in a wide variety of livestock such as pigs, poultry and ruminants, such as sheep and cattle. In particular the invention relates to the prevention and/or treatment of pecking in poultry, in particular in galliformes, and to the prevention and/or treatment of caudophagy in pigs. Galliformes are preferably selected in the group consisting of cocks, capons, hens, chickens, guinea fowl, turkeys and their chicks, such as in particular broiler cocks, broiler capons, broiler hens and broiler chickens and laying hens. Pigs are preferably selected in the group consisting of sows, grower pigs and grower piglets. Ruminants are preferably selected in the group consisting of sheep, fattening cattle, reproducer cattle, calves and dairy cows. The invention is also effective for the prevention and/or treatment of cannibalism in game birds and in particular pheasants, red-legged and gray partridges, quail and mallard ducks.

According to the invention, the yeast derivative containing 5′-nucleotides and/or peptides having a molecular weight of at least 1000 daltons, preferably from 1000 to 10,000 daltons, the 5′-nucleotides and/or the peptides derived from yeast, of a molecular weight of at least 1000 daltons, preferably from 1000 to 10,000 daltons, are advantageously administered to the animals by the oral route. They can be administered in different forms or presentations, in particular in solid form—such as in particular on a granular support, such as feed meal, or in the form of a powder, granules, microgranules or particles—or liquid—preferably water-based—, alone or in combination with other ingredients, such as for example one or several other feed ingredients and/or one or several other nutritional ingredients such as one or several dietary minerals and/or one or several vitamins, and/or diluted into the drinking water or to the main feed.

Thus, according to the invention, the yeast derivative, the 5′-nucleotides and/or the peptides on the granular support can be incorporated into feed meal for laying hens or broiler chickens or into feed meal for pigs, and the granule preparation can be incorporated into mixtures based on grain or crushed and/or whole cereals for poultry, such as in particular mixtures for laying hens. The preparation can also be part of the composition of a nutrient for animal feed in extruded form, for example pig feed.

Generally, for the prevention and/or treatment of caudophagy in pigs, the 5′-nucleotides are preferably administered in a quantity of at least 0.1 g per animal per week, preferably at least 0.2 g per animal per week, and more preferably at least 0.4 g per animal per week. The yeast derivative peptides of at least 1000 daltons, advantageously from 1000 to 10,000 daltons, are preferably administered in a quantity of at least 0.15 g per animal per week, preferably at least 0.30 g and more preferably at least 0.60 g per animal per week. For the prevention and/or treatment of caudophagy in pigs, the yeast derivative containing 5′-nucleotides and/or peptides of at least 1000 daltons, advantageously from 1000 to 10,000 daltons, is preferably administered in a weekly quantity of at least 10 mg/kg of live weight, more preferably at least 20 mg/kg of live weight per animal per week, even more preferably at least 50 mg/kg of live weight per animal per week.

Generally, for the prevention and/or treatment of pecking in cocks, hens or chickens and game birds, the 5′-nucleotides are preferably administered in a quantity of at least 0.001 g per animal per week, preferably at least 0.002 g per animal per week, and more preferably at least 0.003 g per animal per week. The yeast derivative peptides of at least 1000 daltons, advantageously from 1000 to 10,000 daltons, are preferably administered in a quantity of at least 0.002 g per animal per week, preferably at least 0.003 g and more preferably at least 0.004 g per animal per week. For the prevention and/or treatment of pecking in cocks, hens or chickens and game birds, the yeast derivative containing 5′-nucleotides and/or peptides of at least 1000 daltons, advantageously from 1000 to 10,000 daltons, is preferably administered in a weekly quantity of at least 5 mg/kg of live weight, more preferably at least 10 mg/kg of live weight per animal per week, more preferably at least 25 mg/kg of live weight per animal per week.

In an advantageous manner, the treatment is carried out for several weeks and preferably comprises two phases characterized by different weekly doses which can be determined according to the pathological state of the animal.

In an advantageous manner, for the treatment of moderate or severe cannibalism in livestock and in particular in pigs, ruminants, poultry and game birds, the 5′-nucleotides, the yeast derivative peptides and/or the yeast derivatives containing 5′-nucleotides and/or peptides of at least 1000 daltons, advantageously from 1000 to 10,000 daltons, are preferably administered in a weekly dose divided into one or more fractions during the week, preferably divided into three administrations during the week. This first phase preferably lasts for one week. The following weeks constitute the second phase of treatment during which the weekly dose can be reduced. Preferably the weekly dose administered during the second phase of treatment is reduced by a factor of three relative to the weekly dose administered during the first phase. The weekly dose of the second phase is preferably divided into one or more fractions during the week, preferably divided into one fraction for the treatment of moderate cannibalism and two fractions for the treatment of severe cannibalism. The duration of the second phase of treatment is at least three weeks for the treatment of moderate cannibalism and at least five weeks for the treatment of severe cannibalism. Preferably, the treatment is continued until the symptoms have disappeared, and can for example be continued for a duration of approximately 26 weeks.

In an advantageous manner, for the treatment of moderate cannibalism in pigs, game birds and poultry, the weekly doses per animal are preferably defined as follows:
for administration of the yeast derivative, the weekly dose is comprised between 30 mg/kg of live weight and 150 mg/kg of live weight, and preferably on the order of 75 mg/kg of live weight during the first phase following which it is comprised between 10 mg/kg of live weight and 50 mg/kg of live weight during the second phase, preferably on the order of 25 mg/kg of live weight.
for administration of peptides of molecular weight of at least 1000 daltons, advantageously from 1000 to 10,000 daltons, the weekly dose is comprised between 30 mg/kg of live weight and 60 mg/kg of live weight, and preferably on the order of 42 mg/kg of live weight during the first phase following which it is comprised between 10 mg/kg of live weight and 20 mg/kg of live weight during the second phase, preferably on the order of 14 mg/kg of live weight.
for administration of 5′-nucleotides, the weekly dose is comprised between 4.5 mg/kg of live weight and 6 mg/kg of live weight, and preferably on the order of 5.25 mg/kg of live weight during the first phase following which it is comprised between 1.5 mg/kg of live weight and 2 mg/kg of live weight during the second phase, preferably on the order of 1.75 mg/kg of live weight.

In an advantageous manner, for the treatment of moderate cannibalism in ruminants, the aforementioned weekly doses for the treatment of pigs and poultry are multiplied by a factor of 4, i.e., they are preferably defined as follows per animal:
for administration of the yeast derivative, the weekly dose is comprised between 120 mg/kg of live weight and 600 mg/kg of live weight, and preferably on the order of 300 mg/kg of live weight during the first phase following which it is comprised between 40 mg/kg of live weight and 200 mg/kg of live weight during the second phase, preferably on the order of 100 mg/kg of live weight.
for administration of peptides of molecular weight of at least 1000 daltons, advantageously from 1000 to 10,000 daltons, the weekly dose is comprised between 120 mg/kg of live weight and 240 mg/kg of live weight, and preferably on the order of 168 mg/kg of live weight during the first phase following which it is comprised between 40 mg/kg of live weight and 80 mg/kg of live weight during the second phase, preferably on the order of 56 mg/kg of live weight.
for administration of 5′-nucleotides, the weekly dose is comprised between 18 mg/kg of live weight and 24 mg/kg of live weight, and preferably on the order of 21 mg/kg of live weight during the first phase following which it is comprised between 6 mg/kg of live weight and 8 mg/kg of live weight during the second phase, preferably on the order of 7 mg/kg of live weight.

The aforementioned doses useful for the treatment of moderate cannibalism are doubled for the treatment of severe cannibalism. Thus, in an advantageous manner, for the treatment of severe cannibalism in pigs, game birds and poultry, the weekly doses per animal are preferably defined as follows:
for administration of the yeast derivative, the weekly dose is comprised between 60 mg/kg of live weight and 300 mg/kg of live weight, and preferably on the order of 150 mg/kg of live weight during the first phase following which it is comprised between 20 mg/kg of live weight and 100 mg/kg of live weight during the second phase, preferably on the order of 50 mg/kg of live weight.
for administration of peptides of molecular weight of at least 1000 daltons, advantageously from 1000 to 10,000 daltons, the weekly dose is comprised between 60 mg/kg of live weight and 120 mg/kg of live weight, and preferably on the order of 84 mg/kg of live weight during the first phase following which it is comprised between 20 mg/kg of live weight and 40 mg/kg of live weight during the second phase, preferably on the order of 28 mg/kg of live weight.
for administration of 5′-nucleotides, the weekly dose is comprised between 9 mg/kg of live weight and 12 mg/kg of live weight, and preferably on the order of 10.5 mg/kg of live weight during the first phase following which it is comprised between 3 mg/kg of live weight and 4 mg/kg of live weight during the second phase, preferably on the order of 3.5 mg/kg of live weight.

In an advantageous manner, for the treatment of severe cannibalism in ruminants, the aforementioned weekly doses for the treatment of pigs and poultry are multiplied by a factor of four, i.e., they are preferably defined as follows per animal:
for administration of the yeast derivative, the weekly dose is comprised between 240 mg/kg of live weight and 1200 mg/kg of live weight, and preferably on the order of 600 mg/kg of live weight during the first phase following which it is comprised between 80 mg/kg of live weight and 400 mg/kg of live weight during the second phase, preferably on the order of 200 mg/kg of live weight.
for administration of peptides of molecular weight of at least 1000 daltons, advantageously from 1000 to 10,000 daltons, the weekly dose is comprised between 240 mg/kg of live weight and 480 mg/kg of live weight, and preferably on the order of 336 mg/kg of live weight during the first phase following which it is comprised between 80 mg/kg of live weight and 160 mg/kg of live weight during the second phase, preferably on the order of 112 mg/kg of live weight.
for administration of 5′-nucleotides, the weekly dose is comprised between 36 mg/kg of live weight and 48 mg/kg of live weight, and preferably on the order of 42 mg/kg of live weight during the first phase following which it is comprised between 12 mg/kg of live weight and 16 mg/kg of live weight during the second phase, preferably on the order of 14 mg/kg of live weight.

One object of the present invention is also the use of a yeast derivative containing 5′-nucleotides and/or peptides of at least 1000 daltons, preferably from 1000 to 10,000 daltons, for producing a preparation for the prevention and/or treatment of cannibalism in livestock. The present invention further relates to the use of 5′-nucleotides and/or peptides of at least 1000 daltons, preferably from 1000 to 10,000 daltons, for producing a preparation for the prevention and/or treatment of cannibalism in livestock.

In a useful manner, at least one 5′-nucleotide selected in the group consisting of 5′-IMP, 5′-GMP, 5′-AMP, 5′-UMP or 5′-CMP is used for said production. They are preferably used as a combination of at least two of said 5′-nucleotides and more preferably as a combination of three of said 5′-nucleotides, even more preferably as a combination of four or five of said 5′-nucleotides. For example, one can use a combination of 5′-IMP with a 5′-nucleotide selected in the group consisting of 5′-GMP, 5′-AMP, 5′-UMP and 5′-CMP. One can also use a combination of 5′-GMP with a 5′-nucleotide selected in the group consisting of 5′-AMP, 5′-UMP and 5′-CMP. One can also use a combination of 5′-AMP with 5′UMP or 5′-CMP or else a combination of 5′-UMP with 5′-CMP. A combination of 5′-IMP with a combination selected in the group consisting of: 5′-GMP and 5′-AMP, 5′-GMP and 5′-UMP, 5′-GMP and 5′-CMP, 5′-AMP and 5′-UMP, 5′-AMP and 5′-CMP, 5′-UMP and 5′CMP is also possible. Another possibility is a combination of 5′-GMP with a combination selected in the group consisting of: 5′-AMP and 5′-UMP, 5′AMP and 5′CMP, 5′-UMP and 5′-CMP. One can also use a combination of four of said 5′-nucleotides or else a combination of the five said 5′-nucleotides. Preferably, a combination of 5′-GMP, 5′-UMP, 5′-CMP and 5′-AMP; a combination of 5′-GMP, 5′-UMP, 5′-CMP and 5′-IMP; or a combination of 5′-GMP, 5′-UMP, 5′-CMP, 5′-IMP and 5′-AMP is used.

The invention also relates to the use, for producing a preparation for the prevention and/or treatment of cannibalism in livestock, of a yeast derivative, said derivative possibly being an enzymatic hydrolysate of yeast containing non-water soluble membranes and/or membrane fragments of yeast cells, or a yeast extract obtained from an enzymatic hydrolysate of yeast by elimination of said membranes and membrane fragments, and in particular one such yeast derivative containing 5′-nucleotides and/or peptides having a molecular weight of at least 1000 daltons, preferably from 1000 to 10,000 daltons. Said yeast derivatives can be obtained in particular by the aforementioned methods promoting the formation of 5′-nucleotides and/or peptides of molecular weight of at least 1000 daltons.

The yeast preferably belongs to the genus Saccharomyces or Candida. Yeasts belonging to the species Saccharomyces cerevisiae and Candida utilis are preferred. Species belonging to the genus Candida which are food yeasts are often called Torula, Torula therefore being partially synonymous with Candida. In particular the yeast can be a yeast selected in the group consisting of baker's yeasts, brewer's yeasts, wine yeasts and distillery yeasts, and more preferably a baker's yeast. In an advantageous manner, said yeast derivative contains at least 2% by mass, preferably at least 3% by mass, more preferably at least 4% by mass and even more preferably at least 5% by mass of 5′-nucleotides based on dry matter, and/or at least 4% by mass, preferably at least 6% by mass, and even more preferably at least 8% by mass of yeast-derived peptides of molecular weight 1000 to 10,000 daltons based on dry matter.

According to a particularly preferred embodiment, the invention additionally concerns the use, for producing a preparation for the prevention and/or treatment of cannibalism in livestock, of a yeast derivative containing 6.8% of 5′-nucleotides, at least 6% of peptides of molecular weight of 1000 to 10,000 daltons, a total N content determined by the Kjeldahl method of approximately 11.3%, an amino N content determined by Sørensen formol titration method of approximately 4.1% and a protein content (6.25× nitrogen) of approximately 70.6%. Said product, commercialized by Bio Springer (94701 Maisons-Alfort, France), is described in further detail in Table 1.

In particular, the preparation can be a preparation for the prevention and/or treatment of cannibalism in livestock selected in the group consisting of cattle, sheep, pigs, poultry and game birds.

Thus, the preparation can be a preparation for the prevention and/or treatment of caudophagy in pigs, such as in particular sows, grower pigs and grower piglets. It can also be a preparation intended for the prevention and/or treatment of an aggressive behaviour in ruminants, preferably selected in the group consisting of sheep, fattening cattle, reproducer cattle, calves and dairy cows.

The preparation can also be a preparation for the prevention and/or treatment of pecking in game birds and in particular pheasants, red-legged and gray partridges, quail and mallard ducks. The preparation can also be a preparation for the prevention and/or treatment of pecking in poultry, in particular in galliformes such as galliformes selected in the group consisting of cocks, capons, hens, chickens, turkeys, guinea fowl and their chicks, preferably in galliformes selected in the group consisting of broiler cocks, broiler capons, broiler chickens and laying hens.

In a useful manner, the preparation is a preparation for oral administration to livestock.

For instance, the preparation can be a solid preparation, such as a preparation on a granular support, for example a complete feed in the form of feed meal, or in the form of feed pellets, in particular pellets obtained by extrusion, or in the form of a powder, microgranules or particles.

The preparation can also be in liquid form, preferably water-based.

In a preferred manner, the preparation contains the yeast derivative containing 5′-nucleotides and/or peptides of molecular weight of at least 1000 daltons and preferably from 1000 to 10,000 daltons and/or 5′-nucleotides and/or peptides of molecular weight of at least 1000 daltons and preferably from 1000 to 10,000 daltons and is used at doses such as to enable the administration thereof to livestock at the useful weekly doses defined earlier. Preferably, the preparation contains the weekly doses defined earlier for administration of a yeast derivative containing 5′-nucleotides and/or peptides of molecular weight of at least 1000 daltons and preferably from 1000 to 10,000 daltons and/or the useful weekly doses defined earlier for the administration of 5′-nucleotides and/or the useful weekly doses defined earlier for the administration of peptides of molecular weight of at least 1000 daltons and preferably from 1000 to 10,000 daltons. According to a particularly advantageous embodiment, the preparation is used and administered according to the live weight of the animal, in one or more fractions.

The invention also relates to preparations that can be obtained by the methods of production according to the invention, and to the animal feeds and drinks containing them, and to the uses thereof in the preventive or curative treatment of cannibalism. Such feeds products are, for example, feed meal for pigs, feed meal for chicks, chickens or laying hens, mixtures based on grain or crushed and/or whole cereal for poultry and game birds, such as mixtures for laying hens, licking blocks or pecking blocks.

The preparations, feed products and drinks according to the invention can also comprise one or more dietary minerals and/or one or more vitamins.

The invention can be at once preventive, by preventing or delaying the appearance of cannibalism and pecking problems in a livestock facility, and/or curative, by enabling a significant reduction thereof as soon as said problems appear, or even by eliminating said problems. An important advantage of the invention is that the result can be achieved without administering veterinary products to the animals that may leave traces in meat and/or eggs, such as antibiotics in particular.

In a particular embodiment, the livestock cannibalism which is treated and/or prevented according to the invention is not due to stress.

The invention is illustrated in the following examples, which are given for purposes of illustration and not by way of limitation.

### EXAMPLE 1

### Composition of “EXL” Yeast Extract

EXL yeast extract used in examples 2 and 3 is a yeast extract obtained by enzymatic hydrolysis of a yeast of the species Saccharomyces cerevisiae and elimination of the non-water soluble fraction. At 5% in hot water said yeast extract forms a slightly opalescent solution. It is a microgranulate powder allowing dust-free manipulation. Said yeast extract contains approximately 10% by mass of peptides between 1000 daltons and 10,000 daltons.

The main properties of the yeast extract are shown in Table 1.

Yeast extracts of this type are readily available on the market and can be purchased in particular from the company Bio Springer, 94701 Maisons-Alfort, France, it being understood that the leading manufacturers of yeast extracts worldwide offer a yeast extract of this type within their product line of yeast hydrolysates and yeast extracts.

TABLE 1

| Dry matter content (DM) | 96% | by mass |
|---|---|---|
| Total nitrogen (N total as defined above, | 11.3% | by mass |
| Kjeldahl method) | | |
| Amino nitrogen (Amino N as defined above, | 4.1% | by mass |
| Sørenson titration) | | |
| Degradation rate | 36% | |
| Protein (total nitrogen × 6.25) | 70.6% | by mass |
| Fat | 0% | by mass |

Vitamins

| | | |
|---|---|---|
| B1 (thiamin) | 13 | ppm |
| B2 (riboflavin) | 75 | ppm |
| B5 (calcium pantothenate) | 140 | ppm |
| B6 (pyridoxine) | 70 | ppm |
| PP (niacin) | 450 | ppm |
| B12 (cyanocobalamine) | 10 | μg/kg |
| Free amino acids^{a} | 16.05% | by mass |
| Total amino acids^{a} | 55.21% | by mass |
| Total 5′-nucleotides^{a} | 6.8% | by mass |
| 5′-CMP^{a} | 0.68% | by mass |
| 5′-UMP^{a} | 1.95% | by mass |
| 5′-GMP^{a} | 1.97% | by mass |
| 5′-IMP^{a} | 2.20% | by mass |

^{a}quantity determined as percentage by mass on dry matter

### EXAMPLE 2

### Effects of EXL Yeast Extract on the Occurrence of Caudophagy in Pigs

EXL yeast extract was added to the drinking water for three consecutive weeks in a pig fattening unit with a known and recurrent risk of caudophagy. The treatment was based on a weekly dose of 75 mg/kg of live weight administered in three fractions (25 mg/kg of live weight per pig repeated three times a week) during the first week, followed by a weekly dose of 25 mg/kg of live weight per pig given in a single dose.

TABLE 2
Composition and nutritional characteristics
of the feed (% by mass on dry matter)

| Ingredients | % |
|---|---|
| Wheat | 68.4 |
| Soybean oil | 0.9 |
| Soy cake 44 | 19.9 |
| D,L-methionine | 0.109 |
| Calcium carbonate | 8.189 |
| Bicalcium phosphate | 1.742 |
| Premix | 0.400 |

Calculated nutritional composition

| | |
|---|---|
| ME (kcal/kg) | 2650 |
| Protein (%) | 16.8 |
| Available phosphorus (%) | 0.4 |

TABLE 3
Effect of EXL yeast extract on mortality in hens.

| Treatment | Mortality rate (%) |
|---|---|
| Controls | 5.1 |
| EXL | 2.2 |

Supplementation with the yeast extract only took place after caudophagy had been reported. After three weeks, it could be seen that supplementation with EXL yeast extract reduced the occurrence of caudophagy more than two-fold, and this was statistically significant (p<0.05).

### EXAMPLE 3

### Effect of EXL Yeast Extract on Pecking-Induced Mortality in Laying Hens

The effect of EXL yeast extract on mortality in laying hens was evaluated from age 19 to 30 weeks in a factory farm.

800 laying hens (Isa Brown breed) raised in cages at a density of five hens per cage were fed with wheat-based feed the composition of which is shown in Table 2.

The following experimental design was used:

400 laying hens without supplementation (controls),
400 laying hens supplemented with EXL.
   EXL yeast extract was added to the drinking water for 12 weeks on the basis of a theoretical treatment of one weekly dose of 75 mg/kg of live weight divided into three fractions (25 mg/kg of live weight per hen) for the first week followed by a weekly dose of 25 mg/kg per hen administered in a single fraction the following weeks. In practice, this amounts to administering 50 g of yeast extract per 1000 hens.

Sanitary conditions in this test were of good quality.

Table 3 shows the effect of EXL yeast extract on the mortality rate in laying hens. It can be seen that supplementation with EXL yeast extract had a significant positive effect on reducing mortality in laying hens as compared with controls. Inspection of the carcasses showed that EXL yeast extract not only reduced hens mortality, but also led to a considerable reduction in the percentage of carcasses with pecking-induced injuries.

This trial clearly demonstrates that EXL yeast extract is effective at reducing pecking-induced mortality in laying hens.

1-37. (canceled)

38. A method for preventing and/or treating cannibalism in livestock comprising administering a yeast derivative containing 5′-nucleotides and/or peptides of at least 1000 daltons, preferably from 1000 to 10,000 daltons.

39. The method according to claim 38, wherein said yeast derivative contains at least one 5′-nucleotide selected in the group consisting of 5′-IMP, 5′-GMP, 5′-AMP, 5′-UMP and 5′-CMP, alone or in combination(s), preferably in the form of a combination of at least two of said 5′-nucleotides, more preferably a combination of three of said 5′-nucleotides, and even more preferably a combination of four or five of said 5′-nucleotides.

40. The method according to claim 38, wherein the yeast derivative containing 5′-nucleotides and/or peptides of at least 1000 daltons, is an enzymatic hydrolysate of yeast and, preferably, a yeast extract.

41. The method according to claim 40, wherein the yeast belongs to the genus Saccharomyces or Candida, and preferably to the species Saccharomyces cerevisiae or to the species Candida utilis.

42. The method according to claim 40, wherein the yeast is a yeast selected in the group consisting of baker's yeasts, brewer's yeasts, wine yeasts and distillery yeasts, preferably the yeast is a baker's yeast.

43. The method according to claim 40, wherein the yeast derivative contains at least 2% by mass, preferably at least 3% by mass, more preferably at least 4% by mass and even more preferably at least 5% by mass of 5′-nucleotides based on dry matter.

44. The method according to claim 40, wherein the yeast derivative contains at least 4% by mass, preferably at least 6% by mass and even more preferably at least 8% by mass of peptides of molecular weight of 1000 to 10,000 daltons based on dry matter.

45. The method according to claim 40, wherein the livestock are selected in the group consisting of cattle, sheep, pigs, poultry and game birds.

46. The method according to claim 40, wherein the cannibalism is caudophagy in pigs, and preferably in pigs selected in the group consisting of sows, grower pigs and grower piglets.

47. The method according to claim 40, wherein the cannibalism is pecking in poultry, and preferably in galliformes and game birds.

48. The method according to claim 40, wherein the cannibalism is an aggressive behavior in ruminants, and preferably in ruminants selected in the group consisting of sheep, fattening cattle, reproducer cattle, calves and dairy cows.

49. The method according to claim 40, wherein the yeast derivative is administered by oral administration.

50. The method according to claim 49, wherein the yeast derivative is a solid preparation, preferably in the form of a powder or in the form of microgranules or in the form of granules or particles.

51. The method according to claim 49, wherein the yeast derivative is liquid, preferably water-based.

52. A method for preventing and/or treating cannibalism in livestock comprising administering 5′-nucleotides.

53. The method according to claim 52, wherein the 5′-nucleotides are selected in the group consisting of 5′-IMP, 5′-GMP, 5′-AMP, 5-UMP and 5′-CMP, alone or in combination(s), preferably in the form of a combination of at least two of said 5′-nucleotides, more preferably a combination of three of said 5′-nucleotides, and even more preferably a combination of four or five of said 5′-nucleotides.

54. The method according to either one of claims 52 or 53, wherein at least part or preferably all of the 5′-nucleotides originate from a yeast derivative containing 5′-nucleotides, preferably in the form of an enzymatic hydrolysate of yeast containing 5′-nucleotides, and even more preferably in the form of a yeast extract containing 5′-nucleotides.

55. The method according to claim 54, wherein the yeast belongs to the genus Saccharomyces or Candida, and preferably to the species Saccharomyces cerevisiae or to the species Candida utilis.

56. The method according to claim 55, wherein the yeast is a yeast selected in the group consisting of baker's yeasts, brewer's yeasts, wine yeasts and distillery yeasts, preferably the yeast is a baker's yeast.

57. The method according to claim 54, wherein the yeast derivative contains at least 2% by mass, preferably at least 3% by mass, more preferably at least 4% by mass and even more preferably at least 5% by mass of 5′-nucleotides based on dry matter.

58. The method according to claim 54, wherein the yeast derivative additionally contains at least 4% by mass, preferably at least 6% by mass and more preferably at least 8% by mass of peptides of molecular weight of 1000 to 10,000 daltons based on dry matter.

59. The method according to claim 54, wherein the livestock are selected in the group consisting of cattle, sheep, pigs, poultry and game birds.

60. The method according to claim 54, wherein the cannibalism is caudophagy in pigs, and preferably in pigs selected in the group consisting of sows, grower pigs and grower piglets.

61. The method according to claim 54, wherein the cannibalism is pecking in game birds and poultry, preferably in galliformes.

62. The method according to claim 54, wherein the cannibalism is an aggressive behavior in ruminants, and preferably in ruminants selected in the group consisting of sheep, fattening cattle, reproducer cattle, calves and dairy cows.

63. The method according to claim 54, wherein the 5′-nucleotides are administered by oral administration.

64. The method according to claim 63, wherein the 5′-nucleotides are a solid preparation, preferably in the form of a powder or in the form of microgranules or in the form of granules or particles.

65. The method according to claim 63, wherein the 5′-nucleotides are liquid, preferably water-based.

66. A method for preventing and/or treating cannibalism in livestock comprising administering peptides of at least 1000 daltons, preferably from 1000 to 10,000 daltons, derived from yeast.

67. The method according to claim 66, wherein said peptides originate from a yeast derivative, preferably in the form of an enzymatic hydrolysate of yeast, and more preferably in the form of a yeast extract which has optionally undergone ultrafiltration.

68. The method according to claim 67, wherein the yeast belongs to the genus Saccharomyces or Candida, and preferably to the species Saccharomyces cerevisiae or to the species Candida utilis.

69. The method according to claim 68, wherein the yeast is a yeast selected in the group consisting of baker's yeasts, brewer's yeasts, wine yeasts and distillery yeasts, preferably the yeast is a baker's yeast.

70. The method according to claim 67, wherein the yeast derivative contains at least 4% by mass, preferably at least 6% by mass and more preferably at least 8% by mass of peptides of molecular weight 1000 to 10,000 daltons based on dry matter.

71. The method according to claim 67, wherein the yeast derivative also contains at least 2% by mass, preferably at least 3% by mass, more preferably at least 4% by mass and even more preferably at least 5% by mass of 5′-nucleotides based on dry matter.

72. The method according to claim 67, wherein the livestock are selected in the group consisting of cattle, sheep, pigs, poultry and game birds.

73. The method according to claim 67, wherein the cannibalism is caudophagy in pigs, and preferably in pigs selected in the group consisting of sows, grower pigs and grower piglets.

74. The method according to claim 67, wherein the cannibalism is pecking in game birds and poultry, and preferably in galliformes.

75. The method according to claim 67, wherein the cannibalism is an aggressive behaviour in ruminants, and preferably in ruminants selected in the group consisting of sheep, fattening cattle, reproducer cattle, calves and dairy cows.

76. The method according to claim 67, wherein the peptides are administered by oral administration.

77. The method according to claim 76, wherein the peptides are a solid preparation, preferably in the form of a powder or in the form of microgranules or in the form of granules or particles.

78. The method according to claim 76, wherein the peptides are liquid, preferably water-based.
